(19) United States
(12) Patent Application Publication
(10) Pub. No.: US 2005/0043704 A1
(43) Pub. Date: Feb. 24, 2005

(54) Methods of using zonisamide as an adjunctive therapy for partial seizures

(75) Inventor: Ivan Lieberburg, Berkeley, CA (US)

Correspondence Address: Finnegan, Henderson, Farabow,;Garrett & Dunner, L.L.P., 1300 I Street, N.W., Washington, DC, 20005-3315, US

(73) Assignee: Eisai Co., Ltd, Tokyo (JP)

(21) Appl. No.: 10/752,515

(22) Filed: Jan. 8, 2004

Related U.S. Application Data
(63) Continuation-in-part of application No. 10/644,935, filed on Aug. 21, 2003.

(51) Int. Cl. A61M 31/00

(52) U.S. Cl. 604/500; 604/890.1

(57) ABSTRACT

Methods of using zonisamide as an adjunctive therapy for partial seizures are disclosed. In particular, the methods enhance the safety of patients taking pharmaceutical formulations of zonisamide by providing information that increases the awareness of pancreatitis as a possible side effect; wherein the patients and/or prescribing physicians and other medical care providers are advised to monitor for pancreatitis and employ methods that will improve the therapeutic outcome in the few patients who experience pancreatitis associated with zonisamide therapy.

This application is a continuation-in-part of U.S. application Ser. No. 10/644,935, filed on Aug. 21, 2003, which is herein incorporated by reference.

### FIELD OF THE INVENTION

The present invention relates to methods of improving the safety of patients who are receiving administrations of zonisamide (3-benzisoxazole methylene sulfonamide) and those who are in need of zonisamide therapy.

### BACKGROUND OF THE INVENTION

In the United States, over 2 million serious adverse drug reactions (ADRs) occur ever year, with 100,000 associated deaths. This places ADRs as the fourth leading cause of death, ranking ahead of pulmonary disease, diabetes, AIDS, pneumonia, accidents, and automobile deaths. Compounding this problem is the fact that ADRs increase exponentially in patients who take four or more medications concurrently. (See http://www.fda.gov/cder/drug/drugReactions/default.htm, last checked Aug. 20, 2003.)

Most drugs are approved by a Food and Drug Administration review process after an average of 1,500 patient exposures. Clinical trials involving this number of subjects (both healthy volunteers and patients in need of the therapeutic effect of the drug under review) provide a statistically relevant sample of the population from which an assessment of safety and efficacy can be evaluated. However, some drugs have very rare toxicity profiles. Bromfenac, for example, causes hepatotoxicity in 1 out of 20,000 patients. For drugs with rare toxicity, more than 100,000 patients must be exposed to generate a warning signal for the adverse event. In instances where an adverse event is identified in association with a human therapeutic, government regulations require a post-approval follow-up after the drug has been taken to market.

Examples of very serious post-marketing events that have been identified in the recent past include Fen-Phen (fenfluramine—phentermine combination therapy) for weight loss and Rezulin (troglitazone) for diabetes, both of which were later removed from the market because the ADR risks outweighed the therapeutic benefits. Statistical and clinical analysis of large adverse event databases collected by post-marketing surveillance is one method by which identification of the rarer ADRs can be made. For more background on the occurrence and identification of ADRs see, for example, Lazarou, J. et al. JAMA 279(15): 1200-1205 (1998), and Gurwitz, J. H. et al. Am J. Med. 109(2): 87-94 (2000). For a discussion of techniques and difficulties inherent in identifying ADRs in adjunctive therapies of epileptic seizures, see French, J. Epilepsia 43(9): 951-955 (2002), which is hereby incorporated by reference in its entirety.

While Rezulin and Fen-Phen are notable for their extreme and potentially irreversible nature, other adverse drug reactions can be minimized or more easily reversed if they are recognized early, and appropriate and timely medical intervention is made. A few examples of frequently reversible adverse events are cardiac arrhythmias, liver function abnormalities, and irregularities in hematopoiesis. Thus, there remains a need for methods for identifying, for detecting and for treating adverse events associated with drug therapy, in a timely and informed manner.

### SUMMARY OF THE INVENTION

Unexpectedly, it has been found that zonisamide therapy in a very small percentage of patients (available estimates in the United States are about one in seven thousand four hundred fifty-five (1:7,455)) can precipitate acute pancreatitis. It also has been found that by curtailing (either by removal, reduction, or tapering off) the administration of zonisamide dosing, alone or in conjunction with other concomitant medications, alleviation and minimization of this severe adverse event is possible. This is particularly the case when medical intervention to manage the disease and/or removal, reduction, or tapering off of zonisamide is instituted rapidly.

Accordingly, the present invention is directed to methods of using zonisamide for a regulatory agency approved use (e.g., as an adjunctive therapy for partial seizures). The methods improve the safety of zonisamide therapy for patients receiving administrations of the drug, or those who are in need of zonisamide therapy.

In some embodiments, the methods of using zonisamide as an adjunctive therapy for partial seizures improves the safety and health of patients taking zonisamide by increasing the awareness of the patient or patient's guardian that pancreatitis is a possible side effect. Accordingly, a patient may be provided with a therapeutically effective amount of zonisamide, and the patient or the patient's guardian may be informed that abdominal pain, hypovolemia, shock, nausea, anorexia, vomiting, or abdominal distention are symptoms of pancreatitis that require prompt medical evaluation if such symptoms are experienced by the patient. As a result, the patient or patient's guardian can self-monitor for signs and symptoms of pancreatitis, and seek medical attention if such symptoms occur in order to obtain appropriate tests, diagnosis, and treatment. In some embodiments, the present methods reduce the risk of pancreatitis in patients receiving zonisamide therapy.

In other embodiments, the present invention provides methods of using zonisamide as an adjunctive therapy for partial seizures comprising informing a prescribing physician or other medical professional (e.g., an emergency medical worker) that pancreatitis may result from zonisamide therapy and to monitor a patient who is prescribed zonisamide as an adjunctive therapy for partial seizures for abdominal pain, hypovolemia, shock, nausea, anorexia, vomiting, or abdominal distention. The prescribing physician or other medical professional also may be advised that when abdominal pain, hypovolemia, shock, nausea, anorexia, vomiting, or abdominal distention is observed, an appropriate diagnostic be employed to determine whether pancreatitis is present. In addition, the prescribing physician or other medical professional-may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely pancreatitis, which may occur in some patients who receive zonisamide therapy.

The present methods also include methods of administrating zonisamide as an adjunctive therapy for partial seizures comprising providing packaging that includes a pharmaceutical formulation of zonisamide along with information providing a warning that zonisamide may cause pancreatitis in some patients and that one or more symptoms chosen from the group of abdominal pain, hypovolemia, shock, nausea, anorexia, vomiting, and abdominal distention are potentially signs of pancreatitis; and providing the packaging to a patient who has been prescribed zonisamide.

The medical information provided in any of the above described methods concerning the signs and symptoms of pancreatitis may alternatively be provided in layman's terms, so as to be better understood by patients or non-medical professionals. Those of skill in the medical art are familiar with the various layman's terms that can be used to describe the symptoms of pancreatitis.

Other advantages and uses of the present invention will become apparent to those skilled in the art in studying this disclosure; therefore this recitation is not intended to limit the scope of the claims attached hereto.

### DESCRIPTION OF THE EMBODIMENTS

Zonisamide is an antiseizure drug, chemically classified as a sulfonamide and unrelated to other antiseizure agents. Antiepileptic drugs are commonly abbreviated as “AEDs.” The active ingredient is zonisamide, 1,2-benzisoxazole-3-methanesulfonamide. Zonisamide was approved in 2000 for the adjunctive treatment, i.e., taken in conjunction with one or more other AED, treatment of epilepsy in the United States. It was first introduced in Japan approximately 12 years ago, where it also has been used as monotherapy, i.e., without other AEDs as concomitant therapeutics. Zonisamide is not known to be a hepatic enzyme inducer and has been administered adjunctively with almost all of the other regulatory-approved AEDs either in the United States or abroad.

The precise mechanism(s) by which zonisamide exerts its anti-seizure effect is unknown. Zonisamide may produce antiseizure effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization, thus suppressing hyperexcitablity in epileptic foci. In vitro binding studies have demonstrated that zonisamide binds to the GABA/benzodiazepine receptor ionophore complex in an allosteric fashion, which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity (about 1/50^{th} the inhibition compared to acetazolamide), and this pharmacologic effect is not thought to be a major contributing factor in the anti-seizure activity of zonisamide.

ZONEGRAN® (the human therapeutic pharmaceutical formulation containing zonisamide) is indicated as adjunctive therapy for the treatment of partial seizures in adults and is supplied by prescription in the form of 25, 50, and 100 mg capsules. The capsule may be divided, so as to offer smaller increments in dosage. Recommended dosing is once or twice daily, the recommended daily dose of 100 mg at the initiation of therapy should not be divided. ZONEGRAN® is given orally and can be taken with or without food. While other therapeutic uses of zonisamide have been reported, such as treatment of obesity and eating disorders, treatment of neuropathic pain, prophylaxis of migraine attacks, and treatment of mania, these are not indications approved by the Food and Drug Administration (FDA) in the United States, and so are called “off-label” uses. Off-label uses, which are within the discretion of the prescribing physician to write, are also encompassed in the methods presented herein.

Prescribing physicians are informed in the product insert (which contains prescribing information approved by the FDA) that, because of the long half-life of zonisamide, up to two weeks may be required to achieve steady-state levels upon reaching a stable dose or following dosage adjustment. Although the regimen described below has been shown to be tolerated, the prescriber may wish to prolong the duration of treatment at the lower doses in order to fully assess the effects of zonisamide at steady state, noting that many of the side effects of zonisamide are more frequent at doses of 300 mg per day and above. Although there is some evidence of greater response at doses above 100-200 mg/day, the increase appears small and formal dose-response studies have not been conducted.

The initial dose should be 100 mg daily. After two weeks, the dose may be increased to 200 mg/day for at least two weeks. It can be increased to 300 mg/day and 400 mg/day, with the dose stable for at least two weeks to achieve steady state at each level. Evidence from controlled trials suggests that ZONEGRAN® doses of 100-600 mg/day are effective, but there is no suggestion of increasing response above 400 mg/day.

Adjunctive therapy for partial seizures in adults denotes that these patients are already on other anti-epileptic medications, but that they are continuing to seize at a rate that has been deemed by their treating physician to require additional (add-on) therapy. For a recent review of AEDs currently available to American physicians, their efficacies for particular types of epileptic seizures and associated ADRs, see: Ilo Leppik, Epilepsia 42 (Suppl.4): 1-6 (2001).

The use of multiple anti-epileptic medications in the adjunctive setting increases the likelihood of confluent or interactive ADRs, and also may confuse the treating physician as to the causal agent. For instance, when an attending medical professional is presented with a patient taking a combination of medications and manifesting a particular side-effect, it is difficult to diagnose which of the patient's medications (or combination of medications) is responsible for the observed side effect. Typically, the attending physician must consult the medical literature of known adverse events to identify drug(s) that are most likely to cause the observed side-effects. Known adverse events may also be found in the package drug inserts for each drug. The drug(s) having the higher likelihood of causing the observed side-effects are usually reduced or withdrawn first. When such options are exhausted, the patient may have to be systematically withdrawn from the various drugs until the cause is identified. Since zonisamide is typically prescribed as an adjunctive therapy, it presents such complications when side-effects occur.

This situation is further complicated when side-effects occur that are not normally associated with a particular drug. A number of commonly prescribed medications are known to be associated with incidents of pancreatitis. Table 1 provides a sampling of such drugs, along with the language from the package drug inserts for those products. While some drugs, such as valproate (DEPAKOTE®, valproate (sodium salt): valproic acid 1:1), have been associated with numerous incidents of pancreatitis, including resultant fatalities, zonisamide has not been known to cause pancreatitis in patients receiving ZONEGRAN® therapy.

Given this knowledge of adverse events, a medical professional would not suspect zonisamide to be responsible for causing pancreatitis in a patient exhibiting the relevant symptoms. Consequently, the attending medical professional would have no obvious reason to withdraw such a patient from zonisamide, and would allow the therapy to continue while searching for other sources of the pancreatitis. However, a careful review of the data generated in American clinical trials, as well as in ADR reports gathered once commercial marketing began, has yielded the discovery that zonisamide may independently induce acute pancreatitis (AP) in a small number of patients, and has implicated AP in patients receiving zonisamide as an adjunctive therapy. Accordingly, the present invention is directed to methods of increasing the safety of zonisamide therapy in view of its newly discovered role in pancreatitis.

Acute pancreatitis (AP) is defined as an acute inflammatory process of the pancreas with variable involvement of peripancreatic tissues or remote organ systems. A review article containing the current classification, definition and terminology, epidemiology and etiology, pathogenesis and pathological findings, clinical and laboratory findings, and as well as more modern techniques of pancreatic imaging and the associated findings, with emphasis on cross-sectional imaging modalities such as ultrasound, computed tomography, and magnetic resonance imaging can be found in Merkle, Elmar M. et al., European Radiology (Germany) 12 (8) p. 1979-92 (August 2002), which is hereby incorporated by reference in its entirety.

Acute pancreatitis causes pathologic changes in the pancreas ranging from a mild edematous process to an overwhelming necrotizing lesion, which may be fatal. While its symptoms are variable, it is principally characterized by epigastric pain radiating to either the upper quadrant or directly through to the back, and frequently shock develops due to circulating vasoactive substances or retroperitoneal hemorrhage. The typical pain is gnawing, of sudden onset, of exceeding severity, unremitting, and sometimes colicky in character. It is not relieved by vomiting, which is another symptom of pancreatitis, and is little affected by morphine, for example. Other symptoms common in pancreatitis are nausea, anorexia and shock (also referred to as hypovolemia).

Laboratory tests from fluids (serum, urine, ascites) sampled from a patient can be used as diagnostics for AP. Patients with this condition are also usually found to have persistent, high amylase levels in the blood and urine, as well as high lipase levels in the blood. Elevated levels of serum amylase of more than three times the upper limit of normal, and/or a urinary amylase concentration over twice the upper limit of normal, is taken as a decisive indicator of AP (absent overt salivary gland disease and gut perforation or infarction). Serum amylase activity of more than three times the upper limit of normal, and/or a urinary amylase concentration over twice the upper limit of normal, are taken as indicative. After 48 to 72 hours, even with continuing evidence of pancreatitis, total serum amylase levels tend to return to normal; therefore, serum lipase levels are measured for elevated levels concomitantly in making a diagnosis. Serum lipase activity increases in parallel with amylase activity, and measurement of both increases the diagnostic yield.

Other means of diagnosing pancreatitis are available and known to those of skill in the art. These other means and methods include, but are not limited to, imaging techniques, such as computerized tomography (CT scans) with or without contrast enhancing agents, ultrasound and nuclear magnetic resonance, and other lab tests, such as measurement of elevated serum trypsin, or urine amylase:creatine clearance ratio (Cₐₘ/C_{cr}), evidence of leukcoytosis, and a hematocrit of over 50% owing to loss of plasma into the peritoneum.

A CT scan, especially a contrast-enhanced dynamic CT scan (CECT), provides valuable information to the treating physician on the severity and prognosis of AP. In particular, CECT allows estimation of the presence and extent of pancreatic necrosis. Studies suggest that the likelihood of prolonged pancreatitis or serious complication is negligible when the CT reveals an image that scores in the severity ranking index of 1 to 2 and is low with scores of 3 to 6. However, patients with scores of 7 to 10 had 92 percent morbidity and 17 percent mortality.

It was noted as early as 1966 that dogs suffering from an experimental model of severe pancreatitis did not die as frequently if their abdomen was washed with Ringer's lactate to remove the pancreatic-associated ascitic fluid (PAAF). (See Rogers, R. E., et al., Am. J. Surgery 111 (6): 792-4 (1966).) Ascitic fluid is a serous effusate that accumulates in the abdominal cavity, in the present application, as a result of AP. It was suggested that peritoneal lavage removed some toxic substance(s) within the PAAF. During the 1970s and early 1980s, researchers conducted experiments to attempt to determine the factor or factors present in the pancreatic ascites, which was responsible for the systemic effects seen during acute pancreatitis. Their studies showed that a substance present in PAAF was responsible for the hemoconcentrating effect, as well as hypotension seen during severe AP attacks (Ellison et al., J. Surg. Res. 30(3): 241-8 (March 1981)). They subsequently demonstrated that adult respiratory distress syndrome (ARDS) could be induced when the lungs of healthy animals were lavaged with small amounts of PAAF. Hepatic mitochondrial respiration and oxygen consumption was diminished in vitro when hepatic cells were exposed to PAAF. This toxin, therefore, was not specific for one cell or tissue type; in fact, it had profound effects on all organ systems examined. These findings demonstrate the potentially life-threatening sequelae of acute pancreatitis that is allowed to progress. Thus, it is of great importance to begin supportive treatment of patients exhibiting signs and symptoms of AP as quickly as possible.

A standard traditional rationale in treating AP is to “set the gland to rest.” This method of treatment is implemented by restricting the intake of food, administering fluids, and maintaining electrolyte balance in afflicted patients. When diagnosed, the severity of the disease is usually rated as mild (abdominal pain and tension), moderate (tension with guarding and paralytic ileus), or severe (paralytic ileus with diffused peritonitis and/or shock). The level of severity determines the type of medical treatment necessary to support the patient. The more severe the disease the closer the monitoring and medical intervention is required.

In most patients (approximately 85 to 90 percent) with acute pancreatitis, the disease is self-limited and subsides spontaneously, usually within three to seven days after treatment is instituted. Patient and/or guardians should be warned that abdominal pain, nausea, vomiting, and/or anorexia can be symptoms of pancreatitis that require prompt medical evaluation.

If a patient develops AP while on zonisamide therapy, the treating physician should search for other causes of AP. Should no other obvious causes be identified, zonisamide should ordinarily be removed, reduced, or alternatively tapered down to an acceptable level, and alternative treatment for the underlying medical condition may be initiated as clinically indicated. If another cause for the attack is identified, e.g., ethanol, pancreatic duct obstruction, etc., then it would be possible to carefully rechallenge with zonisamide once the acute attack of AP has subsided. If the patient again appears to be developing AP or is diagnosed with AP, then switching to another AED may be warranted.

In patients experiencing acute abdominal pain, or in instances where AP is suspected, the patient's serum amylase levels should be estimated promptly; if these levels are elevated and no other cause is obvious, then the drug should typically be withdrawn or titrated down to a level where the side-effect is no longer a concern. Serum amylase levels should be monitored, as needed, as such symptoms persist and/or subside.

In some cases, it may be possible to reduce or taper-off the level of zonisamide to avoid AP or other side-effects, while maintaining the therapeutic efficacy of the drug therapy. Such decisions may be made by an attending medical personnel, for example, after considering the severity of the AP or other side effects in relation to the patient's need for continued zonisamide therapy.

Conventional support measures for AP of mild severity include (1) analgesia for pain; (2) intravenous fluids and colloids to maintain normal intravascular volume; (3) no oral intake of foods; and (4) optional nasogastric suction to decrease gastrin release from the stomach and prevent gastric contents from entering the duodenum may also be implemented. For moderate to severe AP, the same treatments apply but are increased. Augmenting this supportive treatment in moderate to severe AP is: obligatory use of nasogastric suction in severe cases; and treatment with antibiotics if infection is apparent or if there is extensive pancreatic necrosis.

Other complications must be treated as they arise, and a skilled physician of emergency or internal medicine knows such treatments. For example, abruptly removing anti-epileptic drug therapy from an epileptic patient may result in more severe or more frequent seizures or status epilepticus. Therefore, removal of zonisamide therapy carries the risk of more severe seizures. However, a hospital physician or emergency medical personnel will have access to other pharmacological interventions for short-term control of generalized seizure activity such as either intravenous lorazepam, at a dose of 0.1 mg/kg, or diazepam at 0.2 mg/kg. If sedatives prove insufficient, then a patient also may be administered fosphenyloin, or in status epilepticus, phenobarbital, with careful monitoring for respiratory depression. Intravenous administration is preferred since this route will provide the most rapid attainment of therapeutic serum levels. Additionally, at the treating physician's discretion, an alternate AED may be substituted for zonisamide.

Prevalence In Zonisamide Treated Patients:

The pharmacovigilance data that were collected, reviewed, and analyzed provided the following information in respect of the incidence of AP in the zonisamide-treated patient population. A total of 11 cases fulfilled the criteria of potential pancreatitis cases. These cases were reviewed in detail for evaluation of possible safety signals.

All 11 cases fulfilled serious criteria. Of these 11 cases, ten (10) cases were reported as pancreatitis and one (1) case was reported as amylase and lipase increase.

For Adverse Events Reported as Pancreatitis:

All of the ten (10) pancreatitis cases originated in the United States. Of the ten (10) cases, three (3) were pediatric cases, six (6) were adult cases, and one (1) was of unknown age. Of the ten (10) cases, four (4) recovered, two (2) were recovering at time of report, three (3) had not recovered, and one (1) had an unknown outcome. None of these events were fatal. The development of pancreatitis occurred between three (3) days and three (3) to four (4) months of the initiation of zonisamide treatment.

Of the ten (10) pancreatitis cases, five (5) cases had strong confounding factors, and seemed to be unrelated to zonisamide, but the possibility of zonisamide involvement could not be completely excluded. Four (4) cases had weak confounding factors, and zonisamide involvement may be possible. One (1) case did not seem to have relevant confounding factors, and zonisamide involvement seems possible.

Based on these data, five (5) cases of pancreatitis occurred during zonisamide treatment with no or only weak confounding factors present. All these cases occurred in the US and there were no cases of pancreatitis from Japanese sources with no or only weak confounding factors reported. This amounts to an estimated incidence of 1:7,455 based upon estimated US exposure. This represents a combined estimated incidence of 1:244,491 based upon the combined estimates of Japanese and US exposure.

For Adverse Events Reported as Amylase and Lipase Increase:

The one (1) case of amylase and lipase increase originated from the United States and involved an adult patient. The outcome of this case is unknown. The development of amylase and lipase increase occurred about 4-5 days after the increase of zonisamide dose from 200 mg to 300 mg daily. The patient had initiated zonisamide treatment about 9 to 10 months before the event onset. This case contains weak confounding factors, and zonisamide involvement may be possible.

Based on this data, this case of amylase and lipase increase occurred during zonisamide treatment with only weak confounding factors present. This case occurred in the US and there were no cases of amylase and lipase increase from Japanese sources with no or only weak confounding factors reported. This amounts to an estimated incidence of 1:37,276 based upon estimated US exposure and 1:1,222,453 based upon the combined estimates of Japanese and US exposure.

Estimates:

Estimates of exposure, based upon retail and mail order prescriptions, indicate that the number of unique patients taking zonisamide capsules in the U.S. is about 37,276 (total prescriptions per year/average number of prescriptions per patient per year less a calculated percentage decrease based on estimated annual dropouts) in the time between approval in 2000 and December 2002. Hospital patient data for that period, however, is not available and is not reflected in the estimates. Estimates of patient exposure for Japan indicate that the number of unique patients taking zonisamide is about 1,185,177 for time beginning with the approval in Japan through December 2002. Japanese data includes prescription and hospital patient data. Exposure from clinical trials are not included in the U.S. or Japanese exposure estimates. Based on these statistics, the estimated number of patients exposed to zonisamide in the U.S. and Japan is 1,222,453 unique patients. This is a rather conservative estimate, assuring that the number of patients actually exposed to zonisamide is unlikely to be higher than the estimate provided. Similarly, the incidences of pancreatitis estimated herein are unlikely to be higher than calculated.

Thus, based on the above information, the overall estimated incidence of pancreatitis and amylase/lipase increase in the US exposed population is 1:6,213, based on combining the reported cases of pancreatitis and amylase/lipase increase. There were no cases of pancreatitis and amylase/lipase increase from Japanese sources. This represents a combined estimated incidence of 1:203,742 based upon the combined estimates of Japanese and US exposure.

The following examples are provided to support the practice of the present invention and are not meant and should not be construed to limit the scope of the claims appended hereto.

### EXAMPLE 1

A 40-year old patient experienced acute pancreatitis and an elevated DILANTIN® (phenyloin) plasma level during the use of ZONEGRAN®. The patient had been administered ZONEGRAN® 400 mg daily and DILANTIN® 600 mg daily for the past 3 to 4 months. The patient was hospitalized with symptoms of DILANTIN® toxicity (plasma level of 24 to 25 mcg/ml), amylase and lipase levels in the 2000's U/L, abdominal discomfort, and nausea. The patient was diagnosed with acute pancreatitis (AP); however, a gastroenterology work-up could not identify a cause for the AP. The DILANTIN® dose was reduced and the patient was tapered off ZONEGRAN®. Subsequently, the patient's amylase and lipase levels decreased. The fact that the patient's AP subsided while still on phenyloin (at reduced doses) but only after zonisamide was tapered off, would indicate that zonisamide was the offending agent in this instance.

### EXAMPLE 2

An 83-year-old female patient receiving zonisamide for treatment of neuropathic pain developed difficulty breathing, fever, disorientation/confusion, kidneys “not working well,” irregular heart rate, elevated heart rate, elevated glucose level, and pancreatitis during the use of ZONEGRAN® for neuropathy of her feet.

The patient was hospitalized for the treatment of pneumonia. While hospitalized, she complained of neuropathy described as a burning sensation in her feet, and soon after, ZONEGRAN® at 100 mg daily was initiated. The following day the patient experienced a fever and was disorientated and confused. After several days she was having difficulty breathing, her kidneys were “not working well,” developed an irregular heart rate (the patient reported a heart rate in the 150's), and increased glucose levels. ZONEGRAN® was discontinued on that same day and the patient was placed on oxygen and transferred to the intensive care unit (ICU). She underwent dialysis and later was diagnosed with pancreatitis. Since the concomitant medicines she received (NEURONTIN®, clonidine and ELAVIL®) are not associated with pancreatitis, the likely cause of the attack was the initiation of zonisamide therapy.

While this invention has been described with respect to various specific examples and embodiments, it is to be understood that the invention is not limited thereby and should only be construed by interpretation of the scope of the appended claims.

1. A method of using zonisamide as an adjunctive therapy for partial seizures to improve the safety of such therapy comprising:
providing a patient with a therapeutically effective amount of zonisamide, and
informing the patient or the patient's guardian during the course of zonisamide therapy that abdominal pain, hypovolemia, shock, nausea, anorexia, vomiting, or abdominal distention are symptoms of pancreatitis that require prompt medical evaluation if such symptoms are experienced by the patient.

2. The method of claim 1, wherein the therapeutically effective amount of zonisamide is from 25 mg to 600 mg.

3. The method of claim 2, wherein the therapeutically effective amount of zonisamide is provided in unit dose form.

4. The method of claim 1, wherein the therapeutically effective amount of zonisamide is provided in a unit dose form and in multiple doses to provide for a course of therapy.

5. The method of claim 4, wherein the unit dose is from 25 mg to 200 mg.

6. A method of using zonisamide as an adjunctive therapy for partial seizures to improve the health of a patient receiving such therapy comprising:
providing a patient with a therapeutically effective amount of zonisamide, and
informing the patient or the patient's guardian during the course of such therapy that abdominal pain, hypovolemia, shock, nausea, anorexia, vomiting, or abdominal distention are symptoms of pancreatitis that require prompt medical evaluation if such symptoms are experienced by the patient.

7. The method of claim 6, wherein the therapeutically effective amount of zonisamide is from 25 mg to 600 mg.

8. The method of claim 7, wherein the therapeutically effective amount of zonisamide is provided in unit dose form.

9. The method of claim 6, wherein the therapeutically effective amount of zonisamide is provided in a unit dose form and in multiple doses to provide for a course of therapy.

10. The method of claim 9, wherein the unit dose is from 25 mg to 200 mg.

11. A method of using zonisamide as an adjunctive therapy for partial seizures to reduce the risk of pancreatitis in a patient receiving such therapy comprising:
providing the patient with a therapeutically effective amount of zonisamide, and
informing the patient or the patient's guardian during the course of zonisamide therapy that abdominal pain, hypovolemia, shock, nausea, anorexia, vomiting, or abdominal distention are symptoms of pancreatitis that require prompt medical evaluation if such symptoms are experienced by the patient.

12. The method of claim 11, wherein the therapeutically effective amount of zonisamide is from 25 mg to 600 mg.

13. The method of claim 12, wherein the therapeutically effective amount of zonisamide is provided in unit dose form.

14. The method of claim 11, wherein the therapeutically effective amount of zonisamide is provided in a unit dose form and in multiple doses to provide for a course of therapy.

15. The method of claim 14, wherein the unit dose is from 25 mg to 200 mg.

16. A method of using zonisamide as an adjunctive therapy for partial seizures comprising:
enhancing the safety profile of zonisamide by informing a prescribing physician that pancreatitis may result from zonisamide therapy and to monitor a patient who is prescribed zonisamide as an adjunctive therapy for partial seizures for abdominal pain, hypovolemia, shock, nausea, anorexia, vomiting, or abdominal distention;
recommending that, when abdominal pain, hypovolemia, shock, nausea, anorexia, vomiting, or abdominal distention is observed, an appropriate diagnostic be employed by the physician to determine whether pancreatitis is present; and
recommending that the physician remove, reduce, or taper off zonisamide dosing in the patient and initiate appropriate supportive therapy.

17. The method of claim 16, wherein the diagnostic comprises measurement of serum lipase and amylase levels.

18. The method of claim 16, wherein the diagnostic comprises a contrast-enhanced dynamic computerized tomography (CECT).

19. A method of using zonisamide as an adjunctive therapy for partial seizures comprising:
improving patient outcome by informing an emergency medical worker that a patient who is receiving zonisamide as an adjunctive therapy for partial seizures and exhibits abdominal pain, hypovolemia, shock, nausea, anorexia, vomiting, and abdominal distention may be suffering from pancreatitis; and
recommending performance of an appropriate diagnostic to determine whether pancreatitis is present,
and if pancreatitis is present, recommending that the worker initiate appropriate supportive therapy and discontinue zonisamide dosing in the patient.

20. The method of claim 19, wherein the diagnostic comprises measurement of serum lipase and amylase levels.

21. The method of claim 19, wherein the diagnostic comprises a contrast-enhanced dynamic computerized tomography (CECT).

22. A method of using zonisamide as an adjunctive therapy for partial seizures comprising:
providing packaging that includes a pharmaceutical formulation of zonisamide along with information providing a warning that zonisamide may cause pancreatitis in some patients and that one or more symptoms chosen from the group of abdominal pain, hypovolemia, shock, nausea, anorexia, vomiting, and abdominal distention are potentially signs of pancreatitis; and
providing the packaging to a patient who has been prescribed zonisamide.

23. A method of using zonisamide as an adjunctive therapy for partial seizures comprising:
enhancing the safety of zonisamide by packaging a pharmaceutical formulation of zonisamide along with information providing a warning that zonisamide may cause pancreatitis in some patients and that one or more symptoms chosen from the group of abdominal pain, hypovolemia, shock, nausea, anorexia, vomiting, and abdominal distention are potentially signs of pancreatitis and providing such packaging to a patient who has been prescribed zonisamide therapy.

24. A method of using zonisamide as an adjunctive therapy for partial seizures comprising:
administering a therapeutically effective amount of zonisamide to a subject in need of treatment;
observing the subject for the appearance of at least one symptom of acute pancreatitis; and
if at least one symptom of acute pancreatitis is observed, reducing or tapering off the dosage of the zonisamide to a dosage that does not produce the at least one symptom of acute pancreatitis.

25. The method of claim 24, wherein if at least one symptom of acute pancreatitis is observed, administration of zonisamide is ceased.

26. The method of claim 24, wherein if at least one symptom of acute pancreatitis is observed, the patient is tested for acute pancreatitis.

27. The method of claim 26, wherein the testing comprises at least one of clinical blood tests, computerized tomography, ultrasound, and nuclear magnetic resonance imaging.

28. The method of claim 25, further comprising administering a therapeutically effective amount of zonisamide after at least one symptom of acute pancreatitis has subsided.

29. The method of claim 24, wherein the therapeutically effective amount of zonisamide is from 25 mg to 600 mg.

30. The method of claim 25, wherein the therapeutically effective amount of zonisamide is provided in unit dose form.

31. The method of claim 30, wherein the therapeutically effective amount of zonisamide is provided in a unit dose form and in multiple doses to provide for a course of therapy.

29. A method of administering zonisamide as an adjunctive therapy for partial seizures comprising:
providing a patient with a therapeutically effective amount of zonisamide and a therapeutically effective amount of at least one other anti-epilepsy drug; and
informing the patient or the patient's guardian that abdominal pain, hypovolemia, shock, nausea, anorexia, vomiting, and abdominal distention are potentially signs of pancreatitis that require prompt medical evaluation if such symptoms are experienced by the patient.

30. The method of claim 29, wherein the patient or patient's guardian is informed by reference to a package drug insert.

31. The method of claim 30, wherein the patient or patient's guardian is informed by reference to a package drug insert.

32. A method of using zonisamide as an adjunctive therapy for partial seizures comprising:
advising a physician prescribing zonisamide to a patient to monitor the patient for one or more symptoms chosen from the group of abdominal pain, hypovolemia, shock, nausea, anorexia, vomiting, and abdominal distention,
recommending that when abdominal pain, hypovolemia, shock, nausea, anorexia, vomiting, or abdominal distention is observed, an appropriate diagnostic be employed by the physician to determine whether pancreatitis is present; and
recommending that the physician remove, reduce, or taper off zonisamide dosing in the patient and initiate appropriate supportive therapy.

33. A method for using zonisamide as an adjunctive therapy for partial seizures prescribed by a physician comprising:
monitoring a patient who is receiving administrations of zonisamide for one or more symptoms chosen from the group of abdominal pain, hypovolemia, shock, nausea, anorexia, vomiting, or abdominal distention;
if one or more of said symptoms are observed, determining whether pancreatitis is present in the patient; and
if pancreatitis is diagnosed, reducing or tapering off the zonisamide dosing until the patient's symptoms have subsided.

34. The method of claim 33, wherein the zonisamide dosing is increased after the patient's symptoms have subsided.

35. A method of using zonisamide as an adjunctive therapy for partial seizures prescribed by a physician comprising:
monitoring a patient who is receiving administrations of zonisamide for one or more symptoms chosen from the group of abdominal pain, hypovolemia, shock, nausea, anorexia, vomiting, or abdominal distention;
if one or more of said symptoms are observed, determining whether pancreatitis is present in the patient; and
if pancreatitis is diagnosed, ceasing the zonisamide dosing until the symptoms of pancreatitis have subsided.

36. The method of claim 35, wherein the zonisamide dosing is restored after the patient's symptoms have subsided.
